(19) United States
(12) Patent Application Publication
(10) Pub. No.: US 2025/0387305 A1
(43) Pub. Date: Dec. 25, 2025

(54) WATER-IN-OIL EMULSION COSMETIC COMPOSITION

(71) Applicant: SHISEIDO COMPANY, LTD., Chuo-ku, Tokyo (JP)

(72) Inventors: Kuanting LIN, Tokyo (JP); Ryushi FUKUHARA, Tokyo (JP); Kei UJIMOTO, Tokyo (JP)

(73) Assignee: SHISEIDO COMPANY, LTD., Chuo-ku, Tokyo (JP)

(21) Appl. No.: 18/879,432

(22) PCT Filed: Jul. 14, 2023
(86) PCT No.: PCT/JP2023/026048

§ 371 Date: Dec. 27, 2024

(30) Foreign Application Priority Data
Jul. 28, 2022 (JP) 2022-120568

(51) Int. Cl. A61K 8/06; A61K 8/27; A61K 8/29; A61K 8/85; A61K 8/86; A61Q 17/04

(52) U.S. Cl. CPC A61K 8/064; A61K 8/27; A61K 8/29; A61K 8/85; A61K 8/86; A61Q 17/04

(57) ABSTRACT

To provide a water-in-oil type emulsion cosmetic composition which has high dispersibility of metal oxide particles in a liquid oil, is less likely to cause caking, and has strong high-temperature stability even when no silicone oil is used or only a small amount of a silicone oil is used.

A water-in-oil type emulsion cosmetic composition includes the following components: surface-treated zinc oxide particles, surface-treated titanium oxide particles, polyhydroxystearic acid, polyricinoleic acid polyglycerol ester, an organically modified clay mineral, an aqueous component, and a liquid oil, wherein a content of the polyhydroxystearic acid is less than 1.0 mass %, and a content of the aqueous component is less than 15 mass %.

### TECHNICAL FIELD

The present invention relates to a water-in-oil type emulsion cosmetic composition.

### BACKGROUND ART

In cosmetics having a UV protection effect, such as sunscreens, in general, metal oxide particles such as titanium oxide particles and zinc oxide particles are used as UV scattering agent particles. In addition, such metal oxide particles are dispersed in a liquid oil and then added to cosmetics in many cases.

Generally, when metal oxide particles are dispersed in a liquid oil, a silicone oil is used as an oil serving as a dispersion medium in many cases in order to obtain a favorable dispersion state.

On the other hand, in recent years, there has been demand for cosmetics that are silicone-free or in which a reduced amount of silicone is used.

For example, PTL 1 discloses a fine metal oxide particle dispersion composition in which fine metal oxide particles are dispersed in a non-silicone oiling agent. More specifically, the fine metal oxide particle dispersion composition of PTL 1 contains the following components (a) to (c): (a) fine metal oxide particles having a particle size of 1 to 100 nm and whose surfaces are treated with trialkoxyalkylsilane, (b) polyhydroxystearic acid, and (c) one, two or more oiling agents selected from the group consisting of hydrocarbon oils and ester oils.

### CITATION LIST

### Patent Literature

[PTL 1] Japanese Patent No. 5770492

### SUMMARY OF INVENTION

### Technical Problem

However, in cosmetics that do not contain a silicone oil or contain a small amount of a silicone oil, there is room for further study regarding powder dispersibility and stability against caking when metal oxide powder and the like are added.

In view of the above circumstances, the present invention provides a water-in-oil type emulsion cosmetic composition which has high dispersibility of a UV scattering agent in a liquid oil, is less likely to cause caking, and has strong high-temperature stability even if no silicone oil is used.

### Solution to Problem

The present invention which achieves the above object is as follows.

### <Aspect 1>

A water-in-oil type emulsion cosmetic composition including the following components:
surface-treated zinc oxide particles,
surface-treated titanium oxide particles,
polyhydroxystearic acid,
polyricinoleic acid polyglycerol ester,
an organically modified clay mineral,
an aqueous component, and
a liquid oil,
wherein a content of the polyhydroxystearic acid is less than 1.0 mass %, and
wherein a content of the aqueous component is less than 15 mass %.

### <Aspect 2>

The composition according to Aspect 1,
wherein the surface-treated zinc oxide particles are one, two or more selected from among silane coupling-treated zinc oxide, silicone-treated zinc oxide, and sugar fatty acid ester-treated zinc oxide particles.

### <Aspect 3>

The composition according to Aspect 1 or 2,
wherein the surface-treated zinc oxide particles are one, two or more selected from among triethoxycaprylylsilane-treated zinc oxide particles, dimethicone- and hydrogen dimethicone-treated zinc oxide particles, and dextrin palmitate-treated zinc oxide particles

### <Aspect 4>

The composition according to any one of Aspects 1 to 3,
wherein the surface of the surface-treated titanium oxide particles is treated with a fatty acid.

### <Aspect 5>

The composition according to any one of Aspects 1 to 4,
wherein a content of the surface-treated titanium oxide particles is less than a content of the surface-treated zinc oxide particles, and
wherein a total content of the surface-treated titanium oxide particles and the surface-treated zinc oxide particles is 30 mass % or more.

### <Aspect 6>

The composition according to any one of Aspects 1 to 5,
wherein the organically modified clay mineral is distearyldimonium hectorite.

### <Aspect 7>

The composition according to any one of Aspects 1 to 6,
wherein the content of the aqueous component is 10 mass % or less.

### <Aspect 8>

The composition according to any one of Aspects 1 to 7,
wherein a total content of the polyhydroxystearic acid and the polyricinoleic acid polyglycerol ester is 1.5 to 10 mass %.

### <Aspect 9>

The composition according to any one of Aspects 1 to 8,
wherein the liquid oil is one, two or more selected from among alkyl fatty acid esters, triglycerides, vegetable oils, and hydrocarbon oils.

### <Aspect 10>

The composition according to any one of Aspects 1 to 9, further including bis-diglyceryl polyacyladipate-2.

### <Aspect 11>

The composition according to any one of Aspects 1 to 10, which is a sunscreen cosmetic composition.

### Advantageous Effects of Invention

According to the present invention, it is possible to provide a water-in-oil type emulsion cosmetic composition which has high dispersibility of a UV scattering agent powder in a liquid oil, is less likely to cause caking, and has strong high-temperature stability even when no silicone oil or only a small amount thereof is used.

### DESCRIPTION OF EMBODIMENTS

Hereinafter, embodiments of the present invention will be described in detail. Here, the present invention is not limited to the following embodiments, and various modifications can be performed within the scope of the gist of the invention.

### <<Water-In-Oil Type Emulsion Cosmetic Composition>>

The water-in-oil type emulsion cosmetic composition of the present invention (hereinafter also referred to as a “composition of the present invention”) is a water-in-oil type emulsion cosmetic composition including the following components:
surface-treated zinc oxide particles,
surface-treated titanium oxide particles,
polyhydroxystearic acid,
polyricinoleic acid polyglycerol ester,
an organically modified clay mineral,
an aqueous component, and
a liquid oil,
wherein a content of the polyhydroxystearic acid is less than 1.0 mass %, and
wherein a content of the aqueous component is less than 15 mass %.

When UV scattering agent particles were dispersed in a liquid oil of a water-in-oil type emulsion cosmetic composition without using a silicone oil, they were dispersed insufficiently, which resulted in poor spreadability of the cosmetic composition. In addition, the inventors conducted extensive studies and found that, although it is possible to improve the dispersibility of UV scattering agent particles by adjusting the dispersing agent used together with the UV scattering agent, a caking phenomenon occurs once a certain dispersion state is reached. In addition, even if the dispersibility and caking resistance of the UV scattering agent particles are improved, the high-temperature stability of the cosmetic composition may be poor.

In this case, the caking phenomenon is a phenomenon in which particles such as metal oxide particles having precipitated due to being left to stand stick together and do not return to their original dispersion state, even if subjected to shaking.

Thus, the inventors conducted further extensive studies, and obtained a water-in-oil type emulsion cosmetic composition which has high dispersibility of metal oxide particles, is less likely to cause caking, and has strong high-temperature stability even when no silicone oil or only a small amount thereof is used.

In the present invention, the surface-treated zinc oxide particles are not particularly limited, and examples thereof include sugar fatty acid ester-treated zinc oxide particles, silane coupling agent-treated zinc oxide particles, silicone-treated zinc oxide particles, and stearic acid-treated zinc oxide particles.

The sugar fatty acid ester-treated zinc oxide particles are not particularly limited, and examples thereof include dextrin palmitate-treated zinc oxide particles, dextrin myristate-treated zinc oxide particles, dextrin (palmitate/hexyldecanoate)-treated zinc oxide particles, and dextrin (palmitate/ethylhexanoate)-treated zinc oxide particles, and among these, dextrin palmitate-treated zinc oxide particles are preferable.

The silane coupling agent-treated zinc oxide particles are not particularly limited, and examples thereof include zinc oxide particles treated with the following silane coupling agent. The silane coupling agent is not particularly limited, and examples thereof include trichlorosilanes such as octadecyltrichlorosilane, triethoxysilanes such as octyltriethoxysilane (OTS), and perfluoroalkylsilanes, and among these, octyltriethoxysilane-treated zinc oxide particles (that is, triethoxycaprylylsilane-treated zinc oxide particles) are preferable.

The silicone-treated zinc oxide particles are not particularly limited, and examples thereof include zinc oxide particles treated with the following silicone treatment agent. The silicone treatment agent is not particularly limited, and examples thereof include dimethylpolysiloxane (i.e., dimethicone), methylhydrogenpolysiloxane, a combination of methylhydrogenpolysiloxane and dimethylpolysiloxane, for example, a combination of dimethicone and hydrogen dimethicone, and the like, but the present invention is not limited thereto.

In addition, as the surface-treated zinc oxide particles, commercially available particles may be used without change, or those obtained by treating the surface of zinc oxide particles with a surface treatment agent may be used.

For example, when the surface-treated zinc oxide particles are dextrin palmitate-treated zinc oxide particles, the zinc oxide particles can be mixed and stirred into a dextrin palmitate solution for a certain time and then filtered to obtain dextrin palmitate-treated zinc oxide particles.

In the present invention, the average primary particle size of the surface-treated zinc oxide particles is not particularly limited, and may be, for example, 5 nm or more, 10 nm or more, or 15 nm or more, and may be 200 nm or less, 100 nm or less, or 50 nm or less. In the present invention, unless otherwise specified, the “average primary particle size” of particles may be determined as a diameter of a circle equivalent to the projected area of primary particles in a TEM image.

The shape of the surface-treated zinc oxide particle is not particularly limited, and examples thereof include a spherical shape, a plate shape, a rod shape, a spindle shape, a needle shape, and an irregular shape.

In the composition of the present invention, the content of the surface-treated zinc oxide particles (a total content when the composition contains a mixture of a plurality of surface-treated zinc oxide particles) is not particularly limited. In order to obtain a higher SPF and inhibit caking, the lower limit value of the content of the surface-treated zinc oxide particles with respect to the entire composition may be, for example, 5.0 mass % or more, 10 mass % or more, 15 mass % or more, 18 mass % or more, or 20 mass % or more, and is preferably 15 mass % or more, 18 mass % or more, or 20 mass % or more. In addition, in order to maintain a favorable dispersion state, the upper limit value of the content of the surface-treated zinc oxide particles with respect to the entire composition may be, for example, 50 mass % or less, 45 mass % or less, 40 mass % or less, 35 mass % or less, 30 mass % or less, 25 mass % or less, or 20 mass % or less, and is preferably 30 mass % or less, 25 mass % or less, or 20 mass % or less.

In addition, in the composition of the present invention, the content of the surface-treated zinc oxide particles is preferably larger than the content of surface-treated titanium oxide particles to be described below in order to better exhibit the effect of the present invention.

In addition, in the composition of the present invention, the total content of the surface-treated zinc oxide particles and the surface-treated titanium oxide particles to be described below is not particularly limited, and in order to obtain a higher SPF, for example, the content may be 5.0 mass % or more, 10 mass % or more, 15 mass % or more, 18 mass % or more, 20 mass % or more, 25 mass % or more, 30 mass % or more, or 33 mass % or more, and in order to maintain a favorable dispersion state, the content may be 55 mass % or less, 50 mass % or less, 45 mass % or less, 40 mass % or less, 35 mass % or less, or 33 mass % or less with respect to the entire composition. In this case, when the composition of the present invention contains a plurality of surface-treated zinc oxide particles, the “total content of the surface-treated zinc oxide particles and the surface-treated titanium oxide particles to be described below” herein refers to a total content of all the surface-treated zinc oxide particles and the surface-treated titanium oxide particles to be described below.

In addition, in the composition of the present invention, the surface-treated zinc oxide particles and the surface-treated titanium oxide particles to be described below can be dispersed in a liquid oil. In order to obtain a higher SPF, the total amount of the surface-treated zinc oxide particles and the surface-treated titanium oxide particles to be described below may be 30 parts by mass or more, 35 parts by mass or more, 40 parts by mass or more, 45 parts by mass or more, 50 parts by mass or more, 55 parts by mass or more, or 60 parts by mass or more based on a total amount of 100 parts by mass of the liquid oil. In addition, the upper limit of the total amount of the surface-treated zinc oxide particles and the surface-treated titanium oxide particles to be described below is not particularly limited, and may be, for example, 200 parts by mass or less, 150 parts by mass or less, or 100 parts by mass or less based on a total amount of 100 parts by mass of the liquid oil. When the composition of the present invention contains both a plurality of surface-treated zinc oxide particles, the “total amount of the surface-treated zinc oxide particles and the surface-treated titanium oxide particles to be described below” herein refers to a total amount of all the surface-treated zinc oxide particles and the surface-treated titanium oxide particles to be described below.

### <Surface-Treated Titanium Oxide Particles>

The composition of the present invention contains surface-treated titanium oxide particles. The surface-treated titanium oxide particles can impart a UV protection effect to the composition of the present invention.

In this case, the surface treatment may be, for example, a surface treatment for hydrophobization.

More specifically, examples of surface treatments for titanium oxide particles include a fatty acid treatment (including a metal soap treatment), a fluorine compound treatment, a pendant treatment, a silane coupling agent treatment, a silicone treatment, a titanium coupling agent treatment, an oiling agent treatment, an N-acylated lysine treatment, a polyacrylic acid treatment, an amino acid treatment, an inorganic compound treatment, a plasma treatment, and a mechanochemical treatment, but the present invention is not limited thereto. Among these surface treatments, in consideration of dispersion stability and the like, a fatty acid treatment, in particular, a stearic acid treatment, a stearic acid soap treatment and the like are preferable. Examples of stearic acid soap treatments include a treatment with a metal salt of stearic acid or a mixture of stearic acid and a metal hydroxide (for example, aluminum hydroxide, etc.), but the present invention is not limited thereto. In addition, in order to inhibit the catalytic activity of titanium oxide, it is preferable to subject the titanium oxide particles to a surface treatment with an inorganic compound, for example, aluminum hydroxide, before a surface treatment with an organic compound such as a fatty acid treatment. Therefore, in a preferable aspect, the surface of the titanium oxide particles is treated with, for example, aluminum hydroxide and fatty acids.

As the surface-treated titanium oxide particles, commercially available particles may be used without change, and those obtained by treating the surface of titanium oxide particles with the above surface treatment agent.

In the present invention, the average primary particle size of the surface-treated titanium oxide particles is not particularly limited, and may be, for example, 5 nm or more, 10 nm or more or 15 nm or more, and may be 200 nm or less, 100 nm or less, or 50 nm or less.

The shape of the surface-treated titanium oxide particle is not particularly limited, and examples thereof include a spherical shape, a plate shape, a rod shape, a spindle shape, a needle shape, and an irregular shape.

In the composition of the present invention, the content of the surface-treated titanium oxide particles is not particularly limited. In order to obtain a higher SPF, the lower limit value of the content of the surface-treated titanium oxide particles with respect to the entire composition may be, for example, 1.0 mass % or more, 2.0 mass % or more, 3.0 mass % or more, 4.0 mass % or more, 5.0 mass % or more, 6.0 mass % or more, 7.0 mass % or more, 8.0 mass % or more, 9.0 mass % or more, 10 mass % or more, or 13 mass % or more. In addition, in order to maintain a favorable dispersion state, the upper limit value of the content of the surface-treated titanium oxide particles with respect to the entire composition may be, for example, 20 mass % or less, 15 mass % or less, or 13 mass % or less.

As described above, in the composition of the present invention, the content of the surface-treated titanium oxide particles is preferably less than the content of the above surface-treated zinc oxide particles (a total content when the composition contains a mixture of a plurality of surface-treated zinc oxide particles).

### <Polyhydroxystearic Acid>

The composition of the present invention contains polyhydroxystearic acid. In particular, the polyhydroxystearic acid has, together with polyricinoleic acid polyglycerol ester to be described below, a function of uniformly dispersing UV scattering agent particles such as the above surface-treated zinc oxide particles and surface-treated titanium oxide particles in a liquid oil and has an effect of imparting spreadability to the composition of the present invention.

Polyhydroxystearic acid is a compound oligomerized by forming ester bonds with hydroxystearic acid, commercially available products include, for example, HS oligomer 600 (commercially available from Hokoku Corporation), and Salacos HS-6C (commercially available from The Nisshin OilliO Group, Ltd.), and these can be used. In addition, the degree of polymerization of polyhydroxystearic acid is not particularly limited, and may be, for example, 4 to 8.

In the composition of the present invention, the content of the polyhydroxystearic acid with respect to the entire composition is less than 1.0 mass %. When the content of the polyhydroxystearic acid is less than 1.0 mass %, it is possible to improve the high-temperature stability of the cosmetic composition. More specifically, the content of the polyhydroxystearic acid may be less than 1.0 mass %, 0.9 mass % or less, 0.8 mass % or less, 0.7 mass % or less, 0.6 mass % or less, 0.5 mass % or less, 0.4 mass % or less, or 0.3 mass % or less with respect to the entire composition, and in order to improve the dispersion effect, the content may be more than 0 mass %, 0.05 mass % or more, 0.1 mass % or more, 0.2 mass % or more, 0.3 mass % or more, 0.4 mass % or more, 0.5 mass % or more, 0.6 mass % or more, or 0.7 mass % or more with respect to the entire composition.

### <Polyricinoleic Acid Polyglycerol Ester>

The composition of the present invention contains polyricinoleic acid polyglycerol ester. In particular, the polyricinoleic acid polyglycerol ester has, together with the above polyhydroxystearic acid, a function of uniformly dispersing UV scattering agent particles such as the above surface-treated zinc oxide particles and surface-treated titanium oxide particles in a liquid oil and has an effect of imparting spreadability as well as caking resistance and high-temperature stability to the composition of the present invention. In addition, the polyricinoleic acid polyglycerol ester also has a function of an emulsifying agent in the composition of the present invention.

More specifically, the polyricinoleic acid polyglycerol ester is not particularly limited, and examples thereof include polyglyceryl-4 polyricinoleate, polyglyceryl-5 polyricinoleate, polyglyceryl-6 polyricinoleate, and polyglyceryl-10 polyricinoleate.

In the composition of the present invention, the content of polyricinoleic acid polyglycerol ester is not particularly limited, and may be, for example, more than 0 mass %, 0.5 mass % or more, 1.0 mass % or more, 1.5 mass % or more, 2.0 mass % or more, 2.2 mass % or more, 2.5 mass % or more, 2.7 mass % or more, or 3.0 mass % or more, and may be 10 mass % or less, 9.0 mass % or less, 8.0 mass % or less, 7.0 mass % or less, 6.0 mass % or less, 5.0 mass % or less, 4.0 mass % or less, or 3.0 mass % or less with respect to the entire composition.

In addition, in the composition of the present invention, in order to better exhibit functions of both a dispersing agent and an emulsifying agent, the content of polyricinoleic acid polyglycerol ester is preferably larger than the content of the polyhydroxystearic acid. In addition, in the composition of the present invention, the total content of polyhydroxystearic acid and polyricinoleic acid polyglycerol ester with respect to the entire composition may be, for example, in a range of 1.5 to 10 mass %. More specifically, the total content of polyhydroxystearic acid and polyricinoleic acid polyglycerol ester may be 1.5 mass % or more, 1.6 mass % or more, 1.7 mass % or more, 1.8 mass % or more, 1.9 mass % or more, 2.0 mass % or more, 2.1 mass % or more, 2.2 mass % or more, 2.3 mass % or more, 2.4 mass % or more, 2.5 mass % or more, 2.6 mass % or more, 2.7 mass % or more, 2.8 mass % or more, 2.9 mass % or more, 3.0 mass % or more, 3.1 mass % or more, 3.2 mass % or more, 3.3 mass % or more, 3.4 mass % or more, 3.5 mass % or more, 3.6 mass % or more, 3.7 mass % or more, 3.8 mass % or more, 3.9 mass % or more, 4.0 mass % or more, 4.1 mass % or more, 4.2 mass % or more, 4.3 mass % or more, 4.4 mass % or more, or 4.5 mass % or more, and may be 10 mass % or less, 9.5 mass % or less, 9.0 mass % or less, 8.5 mass % or less, 8.0 mass % or less, 7.5 mass % or less, 7.0 mass % or less, 6.5 mass % or less, 6.0 mass % or less, 5.5 mass % or less, 5.0 mass % or less, 4.5 mass % or less, or 4.0 mass % or less with respect to the entire composition.

### <Organically Modified Clay Mineral>

The composition of the present invention contains an organically modified clay mineral. When the organically modified clay mineral is contained, it is possible to improve the caking resistance of the composition of the present invention.

The organically modified clay mineral is a type of colloidal hydrous aluminum silicate having a three-layer structure, and a typical example thereof is a clay mineral represented by the following General Formula (1) modified with a quaternary ammonium salt type cationic surfactant.

(X,Y)₂₋₃(Si,Al)₄O₁₀(OH)₂Z_{1/3}·nH₂O (1)

In Formula (1), X is Al, Fe(III), Mn(III), or Cr(III), Y is Mg, Fe(II), Ni, Zn, or Li, and Z is K, Na, or Ca.

Specific examples of organically modified clay minerals include dimethyl distearammonium hectorite (disteardimonium hectorite), dimethyl alkyl ammonium hectorite, benzyl dimethyl stearyl ammonium hectorite, and distearyl dimethyl ammonium chloride-treated magnesium aluminum silicate, but the present invention is not limited thereto. Among these, the organically modified clay mineral is preferably disteardimonium hectorite. In addition, as commercially available organically modified clay minerals, for example, Bentone 27 (benzyl dimethyl stearyl ammonium chloride-treated hectorite: commercially available from Elementis Japan Co., Ltd.), Bentone 38 (distearyl dimethyl ammonium chloride-treated hectorite: commercially available from Elementis Japan Co., Ltd.) and the like can be used.

In the composition of the present invention, the content of the organically modified clay mineral is not particularly limited, and may be, for example, 0.05 mass % or more, 0.1 mass % or more, 0.2 mass % or more, 0.3 mass % or more, 0.4 mass % or more, or 0.5 mass % or more, and may be 10 mass % or less, 5.0 mass % or less, or 1.0 mass % or less with respect to the entire composition.

### <Aqueous Component>

The composition of the present invention contains an aqueous component. The aqueous component can impart a refreshing effect to the composition of the present invention and can improve the caking resistance of the composition of the present invention.

The aqueous component is not particularly limited, and for example, water, monohydric alcohols such as ethanol, polyhydric alcohols such as butylene glycol, dipropylene glycol, propanediol, pentylene glycol, hexanediol, and glycerin, polyalkylene glycol such as polyethylene glycol and polypropylene glycol or copolymers thereof, inorganic salts such as sodium chloride and magnesium chloride, or mixtures thereof can be used.

In the composition of the present invention, the content of the aqueous component with respect to the entire composition is less than 15 mass %. When the content of the aqueous component is less than 15 mass %, it is possible to improve the spreadability and high-temperature stability of the cosmetic composition. More specifically, the content of the aqueous component with respect to the entire composition may be less than 15 mass %, 14 mass % or less, 13 mass % or less, 12 mass % or less, 11 mass % or less, 10 mass % or less, 9.0 mass % or less, 8.0 mass % or less, 7.0 mass % or less, 6.0 mass % or less, 5.0 mass % or less, or 4.0 mass % or less, and in order to improve the refreshing effect and caking resistance, the content may be more than 0 mass %, 0.5 mass % or more, 1.0 mass % or more, or 2.0 mass % or more.

### <Liquid Oil>

The composition of the present invention contains a liquid oil. The liquid oil refers a liquid-like oil having fluidity at room temperature (25° C.) under atmospheric pressure (1 atm (9.8×10⁴ Pa)), and may be, for example, an oil having a melting point of 25° C. or lower (preferably lower than 25° C., 20° C. or lower, 15° C. or lower, or 10° C. or lower).

In the present invention, the liquid oil is not particularly limited, and in order to better exhibit the effect of the present invention, it is preferable that the composition do not contain a silicone oil or contain a small content of silicone oil (for example, a content of 10 mass % or less, 5.0 mass % or less, 1.0 mass % or less, 0.5 mass % or less, 0.1 mass % or less, or 0.01 mass % or less with respect to the entire composition).

In the present invention, the liquid oil is not particularly limited, and may be, for example, one, two or more selected from among alkyl fatty acid esters, triglycerides, vegetable oils, and hydrocarbon oils.

More specifically, examples of alkyl fatty acid esters include palmitates such as octyl palmitate, octanoates such as cetyl octanoate, isooctanoates such as glyceryl tri-2-ethylhexanoate, and pentaerythritol tetra-2-ethylhexanoate, laurates such as hexyl laurate, myristates such as isopropyl myristate and octyldodecyl myristate, stearates such as isocetyl stearate, isostearates such as isopropyl isostearate, isopalmitates such as isooctyl palmitate, oleates such as isodecyl oleate, adipic acid diesters such as diisopropyl adipate, sebacic acid diesters such as diethyl sebacate, and ester oils such as diisostearyl malate, but the present invention is not limited thereto.

Examples of triglycerides include tricaprylin, tricaproin, triisostearin, trielaidin, trierucin, trilinolein, triolein, caprylic/capric triglyceride, caprylic/capric/linoleic triglyceride, and ricinoleic/caproic/caprylic/capric triglyceride, but the present invention is not limited thereto.

Examples of vegetable oils include jojoba seed oil, olive oil, macadamia nut oil, meadowfoam oil, castor oil, safflower oil, sunflower oil, avocado oil, canola oil, apricot kernel oil, rice germ oil, and rice bran oil, but the present invention is not limited thereto.

Examples of hydrocarbon oils include branched hydrocarbon oils such as squalane, isododecane, and isohexadecane, and linear hydrocarbon oils such as undecane and tridecane, but the present invention is not limited thereto.

In the composition of the present invention, the content of the liquid oil is not particularly limited, and may be, for example, 10 mass % or more, 20 mass % or more, 30 mass % or more, 40 mass % or more, 50 mass % or more, or 60 mass % or more, and may be 90 mass % or less, 80 mass % or less, or 60 mass % or less with respect to the entire composition.

### <Optional Components>

The composition of the present invention may further contain, as an optional component, a semi-solid oil such as bis-diglyceryl polyacyladipate-2. In the present invention, since the semi-solid oil is an oil that is not completely solidified or liquefied at room temperature (25° C.) under atmospheric pressure (1 atm (9.8×10⁴ Pa)), it is distinguished from a liquid oil and a solid oil, and has a melting point in a range of, for example, higher than 25° C. (preferably 30° C. or higher) and 55° C. or lower (preferably 50° C. or lower). In the present invention, “solid” refers to an oil that can maintain its solid state at room temperature (25° C.) under atmospheric pressure (1 atm (9.8×10⁴ Pa)).

Bis-diglyceryl polyacyladipate-2 is an ester oil of diglycerol, and adipic acid, octanoic acid, decanoic acid, isostearic acid, stearic acid, or hydroxystearic acid. Bis-diglyceryl polyacyladipate-2 can function as an emollient agent and a water resistance improving agent in the composition of the present invention.

When the composition of the present invention contains bis-diglyceryl polyacyladipate-2, the content thereof is not particularly limited, and may be, for example, 0.1 mass % or more, 0.5 mass % or more, 1.0 mass % or more, 2.0 mass % or more, or 3.0 mass % or more, and may be 10 mass % or less, 5.0 mass % or less, or 3.0 mass % or less with respect to the entire composition.

In addition to the above components, the composition of the present invention may further contain, optionally, for example, alcohols, moisturizing agents, thickeners, powder components (powders other than the above surface-treated zinc oxide particles and surface-treated titanium oxide particles), antioxidants, stabilizing agents, chelating agents, preservatives, and fragrance agents.

### <Preparation Method and Use>

The composition of the present invention can be prepared according to a method generally used for water-in-oil type emulsion cosmetic compositions. For example, the composition can be prepared by separately preparing an oil phase component obtained by dispersing UV scattering agent powder such as the above surface-treated zinc oxide particles and surface-treated titanium oxide particles in a liquid oil, and an aqueous component, adding the aqueous component while stirring the oil phase component, and performing emulsifying.

The composition of the present invention can be a type of cosmetic in which UV scattering agent powder such as the above surface-treated zinc oxide particles and surface-treated titanium oxide particles precipitates due to being left to stand when not in use and UV scattering agent powder is uniformly dispersed again due to shaking when in use.

Therefore, the composition of the present invention can be used as a sunscreen cosmetic. More specifically, the composition of the present invention can be provided as, for example, a sunscreen cream, a sunscreen emulsion, or a sunscreen lotion, and can also be used as a foundation, a makeup base, a makeup cosmetic, a hair cosmetic or the like with a sunscreen effect imparted.

### EXAMPLES

The present invention will be described below in more detail with reference to examples, but the present invention is not limited thereto. Here, unless otherwise specified, the addition amount is mass % based on the mass of the cosmetic composition.

### Examples 1 to 8 and Comparative Examples 1 to 7

Based on the formulations shown in the following Table 1 and Table 2, cosmetic compositions of examples and comparative examples were prepared. The obtained compositions were evaluated for “spreadability,” “caking resistance,” and “high-temperature stability” according to the following criteria, and the results are shown in Table 1.

The compositions obtained in the examples and comparative examples were subjected to a usage test by 20 cosmetic evaluation specialist panelists. Here, the spreadability is due to the dispersion state of metal oxide particles dispersed in each composition. Each panelist evaluated “spreadability” for the compositions on a 5-point scale according to the following “evaluation criteria,” and additionally, the average score of the scores of all the panelists was determined according to the following 4-point scale determination criteria.

### (Evaluation Criteria)

evaluation results: score
very good: 5 points
good: 4 points
average: 3 points
slightly poor: 2 points
poor: 1 point

### (Determination Criteria)

“A”: the average score was 4 or more
“B”: the average score was 3 or more and less than 4
“C”: the average score was 1.5 or more and less than 3
“D”: the average score was less than 1.5

### <Caking Resistance>

Immediately after the compositions obtained in the examples and comparative examples were prepared, the samples were evaluated for caking resistance according to an accelerated precipitation evaluation test.

More specifically, 35 g of each sample was filled into a 50 ml centrifugal precipitation tube, and centrifuged at 800 rpm for 30 minutes. The samples after centrifugation were shaken a predetermined number of times, and determined according to the following evaluation criteria:
“A”: when the precipitated powder was dispersed again and the sample became homogenous after shaking 1 to 10 times,
“B”: when the precipitated powder was dispersed again and the sample became homogenous after shaking 11 to 20 times,
“C”: when the precipitated powder was dispersed again and the sample became homogenous after shaking 21 to 100 times,
“D”: when the precipitated powder was not dispersed again even after shaking 150 times.

Here, in the case of “A,” no caking occurred, that is, the caking resistance was evaluated as being best. In addition, the caking resistance was evaluated as getting worse in the order from “B” to “D,” and particularly in the case of “D,” the caking resistance was evaluated as being worst.

### <High-Temperature Stability>

The compositions obtained in the examples and comparative examples were stored at 50° C. for 4 weeks, and the high-temperature stability was then evaluated according to an accelerated precipitation evaluation test.

More specifically, after storage at 50° C. for 4 weeks, 35 g of each sample was filled into a 50 ml centrifugal precipitation tube, and centrifuged at 800 rpm for 30 minutes. The sample container after centrifugation was shaken a predetermined number of times and determined according to the following evaluation criteria:
“A”: when the precipitated powder was dispersed again and the sample became homogenous after shaking 1 to 10 times,
“B”: when the precipitated powder was dispersed again and the sample became homogenous after shaking 11 to 20 times,
“C”: when the precipitated powder was dispersed again and the sample became homogenous after shaking 21 to 100 times,
“D”: when the precipitated powder was not dispersed again even after shaking 150 times.
“E”: when the sample was strongly thickened and gelled (before applying centrifugation) and lost fluidity, and it was difficult to perform the accelerated precipitation evaluation test.

Here, in the case of “A,” no caking occurred, and the caking resistance was the best, that is, the high-temperature stability after storage at 50° C. for 4 weeks was evaluated as being the best. In addition, the high-temperature stability was evaluated as getting worse in the order from “B” to “E,” and particularly in the case of “E,” the accelerated precipitation evaluation test could not be performed, and the high-temperature stability was evaluated as being worst.

TABLE 1
(Table 1 Examples 1 to 8 and Comparative Examples 1 to 7)

| Constituent | | | | | | | | | | Comparative |
|---|---|---|---|---|---|---|---|---|---|---|
| components | | Example 1 | Example 2 | Example 3 | Example 4 | Example 5 | Example 6 | Example 7 | Example 8 | Example 1 |
| Surface-treated | | 13 | 13 | 13 | 13 | 13 | 13 | 13 | 13 | 13 |
| titanium oxide | | | | | | | | | | |
| particles | | | | | | | | | | |
| (stearic | | | | | | | | | | |
| acid/aluminum | | | | | | | | | | |
| hydroxide/titanium | | | | | | | | | | |
| oxide) | | | | | | | | | | |
| Surface-treated | | 20 | 20 | 20 | 20 | 20 | 20 | — | — | 20 |
| zinc oxide | | | | | | | | | | |
| particles | | | | | | | | | | |
| (dextrin | | | | | | | | | | |
| palmitate/zinc | | | | | | | | | | |
| oxide) | | | | | | | | | | |
| Surface-treated | | — | — | — | — | — | — | 20 | — | — |
| zinc oxide | | | | | | | | | | |
| particles | | | | | | | | | | |
| (triethoxycaprylyl | | | | | | | | | | |
| silane/zinc oxide) | | | | | | | | | | |
| Surface-treated | | — | — | — | — | — | — | — | 20 | — |
| zinc oxide | | | | | | | | | | |
| particles | | | | | | | | | | |
| (dimethicone/hydrogen | | | | | | | | | | |
| dimethicone/zinc | | | | | | | | | | |
| oxide) | | | | | | | | | | |
| Polyhydroxystearic | | 0.5 | 0.7 | 0.3 | 0.5 | 0.5 | 0.5 | 0.5 | 0.5 | 3.3 |
| acid | | | | | | | | | | |
| Polyricinoleic | | 2.7 | 2.7 | 2.7 | 1.8 | 4.0 | 2.7 | 2.7 | 2.7 | — |
| acid polyglycerol | | | | | | | | | | |
| ester | | | | | | | | | | |
| (polyglyceryl-6 | | | | | | | | | | |
| polyricinoleate) | | | | | | | | | | |
| Bis-diglyceryl | | 3.0 | 3.0 | 3.0 | 3.0 | 3.0 | 3.0 | 3.0 | 3.0 | 3.0 |
| polyacyladipate-2 | | | | | | | | | | |
| Aqueous | Water | 1.0 | 1.0 | 1.0 | 1.0 | 1.0 | 2.0 | 2.0 | 2.0 | 1.0 |
| component | Glycerin | 1.0 | 1.0 | 1.0 | 1.0 | 1.0 | 2.0 | 2.0 | 2.0 | 1.0 |

| | | | | | | | | | |
|---|---|---|---|---|---|---|---|---|---|
| Organically | 0.5 | 0.5 | 0.5 | 0.5 | 0.5 | 0.5 | 0.5 | 0.5 | 0.5 |
| modified clay | | | | | | | | | |
| mineral | | | | | | | | | |
| (distearyldimonium | | | | | | | | | |
| hectorite) | | | | | | | | | |
| Liquid oil | remainder | remainder | remainder | remainder | remainder | remainder | remainder | remainder | remainder |
| (Details are shown | | | | | | | | | |
| in Table 2) | | | | | | | | | |
| Sum | 100 | 100 | 100 | 100 | 100 | 100 | 100 | 100 | 100 |
| Spreadability | A | A | A | A | A | A | A | A | A |
| Caking resistance | A | A | A | A | A | A | A | A | D |
| (centrifugal | | | | | | | | | |
| acceleration) | | | | | | | | | |
| High-temperature | A | A | A | A | A | A | A | A | D |
| stability | | | | | | | | | |

| Constituent | Comparative | Comparative | Comparative | Comparative | Comparative | Comparative |
|---|---|---|---|---|---|---|
| components | Example 2 | Example 3 | Example 4 | Example 5 | Example 6 | Example 7 |
| Surface-treated | 13 | 13 | 13 | 13 | 13 | 13 |
| titanium oxide | | | | | | |
| particles | | | | | | |
| (stearic | | | | | | |
| acid/aluminum | | | | | | |
| hydroxide/titanium | | | | | | |
| oxide) | | | | | | |
| Surface-treated | 20 | 20 | 20 | 20 | 20 | 20 |
| zinc oxide | | | | | | |
| particles | | | | | | |
| (dextrin | | | | | | |
| palmitate/zinc | | | | | | |
| oxide) | | | | | | |
| Surface-treated | — | — | — | — | — | — |
| zinc oxide | | | | | | |
| particles | | | | | | |
| (triethoxycaprylyl | | | | | | |
| silane/zinc oxide) | | | | | | |
| Surface-treated | — | — | — | — | — | — |
| zinc oxide | | | | | | |
| particles | | | | | | |
| (dimethicone/hydrogen | | | | | | |
| dimethicone/zinc | | | | | | |
| oxide) | | | | | | |
| Polyhydroxystearic | — | 1.0 | — | 0.5 | 0.5 | 0.5 |
| acid | | | | | | |
| Polyricinoleic | 3.3 | 2.7 | 2.7 | — | 2.7 | 2.7 |
| acid polyglycerol | | | | | | |
| ester | | | | | | |
| (polyglyceryl-6 | | | | | | |
| polyricinoleate) | | | | | | |
| Bis-diglyceryl | 3.0 | 3.0 | 3.0 | 3.0 | 3.0 | 3.0 |
| polyacyladipate-2 | | | | | | |
| Aqueous | 1.0 | 1.0 | 1.0 | 1.0 | 10 | 7.5 |
| component | 1.0 | 1.0 | 1.0 | 1.0 | 10 | 7.5 |
| Organically | 0.5 | 0.5 | 0.5 | 0.5 | 0.5 | 0.5 |
| modified clay | | | | | | |
| mineral | | | | | | |
| (distearyldimonium | | | | | | |
| hectorite) | | | | | | |
| Liquid oil | remainder | remainder | remainder | remainder | remainder | remainder |
| (Details are shown | | | | | | |
| in Table 2) | | | | | | |
| Sum | 100 | 100 | 100 | 100 | 100 | 100 |
| Spreadability | D | A | D | D | D | B |
| Caking resistance | A | D | A | A | A | A |
| (centrifugal | | | | | | |
| acceleration) | | | | | | |
| High-temperature | A | A | A | A | E | B |
| stability | | | | | | |

TABLE 2
(Table 2 Details of “liquid oil” in Table 1)

| | | Addition amount |
|---|---|---|
| | | (mass % based on total |
| Liquid oil | | mass of liquid oil) |
| Vegetable oil | Jojoba seed oil | remainder |
| Branched | Squalane | 20 |
| hydrocarbon oil | | |
| Triglyceride | Tricaprylin | 14 |
| Alkyl fatty acid | Octyl palmitate | 9.0 |
| ester | | |
| Linear hydrocarbon | Undecane | 12 |
| oil | Tridecane | 5.0 |
| Antioxidant | Tocopherol | appropriate amount |

| | |
|---|---|
| Sum | 100 |

As is clear from the results in Table 1, it can be understood that the compositions of Examples 1 to 8 all had excellent spreadability, caking resistance and high-temperature stability in the evaluation results.

On the other hand, it can be understood that, in Comparative Examples 1 and 5 containing no polyricinoleic acid polyglycerol ester, when the content of the polyhydroxystearic acid was increased to 3.3 mass % (Comparative Example 1), the spreadability evaluation result was good, but the caking resistance and high-temperature stability evaluation results were poor. On the other hand, it can be understood that, when the content of the polyhydroxystearic acid was reduced to 0.5 mass % (Comparative Example 5), the spreadability evaluation result was poor.

In addition, it can be understood that, in Comparative Examples 2 and 4 containing no polyhydroxystearic acid, even when the content of polyricinoleic acid polyglycerol ester was changed, the spreadability evaluation result was poor.

In addition, it can be understood that, in Comparative Example 3 in which the content of the polyhydroxystearic acid was 1.0 mass %, the caking resistance and high-temperature stability evaluation results were poor.

In addition, it can be understood that, in Comparative Examples 6 and 7 in which the content of the aqueous component was 15 mass % or more, the caking resistance evaluation result was good, but the spreadability and high-temperature stability evaluation results were both poor.

### Examples 9 to 11 and Comparative Example 8

Compositions of Examples 9 to 11 were prepared in the same manner as in Example 6 except that various polyricinoleic acid polyglycerol esters shown in Table 3 were used in place of polyglyceryl-6 polyricinoleate which is a constituent component of Example 6.

In addition, a composition of Comparative Example 8 was prepared in the same manner as in Example 6 except that, in place of polyglyceryl-6 polyricinoleate, which is a constituent component of Example 6, isostearic acid known as a general surfactant was used, and the content of the aqueous component was added as shown in Table 3.

In the same manner as above, these compositions were evaluated for “spreadability,” “caking resistance,” and “high-temperature stability,” and the results are shown in Table 3. Here, for reference, the results of Example 6 are also shown in Table 3.

TABLE 3
(Table 3 Examples 6 and 9 to 11 and Comparative Example 8)

| | | | | | | Comparative |
|---|---|---|---|---|---|---|
| Constituent components | | Example 6 | Example 9 | Example 10 | Example 11 | Example 8 |
| Surface-treated titanium oxide particles | | 13 | 13 | 13 | 13 | 13 |
| (stearic acid/aluminum hydroxide/titanium | | | | | | |
| oxide) | | | | | | |
| Surface-treated zinc oxide particles | | 20 | 20 | 20 | 20 | 20 |
| (dextrin palmitate/zinc oxide) | | | | | | |
| Polyhydroxystearic acid | | 0.5 | 0.5 | 0.5 | 0.5 | 0.5 |
| Polyricinoleic acid | Polyglyceryl-6 | 2.7 | — | — | — | — |
| polyglycerol | polyricinoleate | | | | | |
| ester | Polyglyceryl-10 | — | 2.7 | — | — | — |
| | polyricinoleate | | | | | |
| | Polyglyceryl-5 | — | — | 2.7 | — | — |
| | polyricinoleate | | | | | |
| | Polyglyceryl-4 | — | — | — | 2.7 | — |
| | polyricinoleate | | | | | |
| Surfactant | Isostearic acid | — | — | — | — | 2.7 |
| Bis-diglyceryl polyacyladipate-2 | | 3.0 | 3.0 | 3.0 | 3.0 | 3.0 |
| Aqueous | Water | 2.0 | 2.0 | 2.0 | 2.0 | 1.0 |
| component | Glycerin | 2.0 | 2.0 | 2.0 | 2.0 | 1.0 |

| | | | | | |
|---|---|---|---|---|---|
| Organically modified clay mineral | 0.5 | 0.5 | 0.5 | 0.5 | 0.5 |
| (distearyldimonium hectorite) | | | | | |
| Liquid oil | remainder | remainder | remainder | remainder | remainder |
| (Details are shown in Table 2) | | | | | |
| Sum | 100 | 100 | 100 | 100 | 100 |
| Spreadability | A | A | A | A | D |
| Caking resistance (centrifugal | A | A | A | A | A |
| acceleration) | | | | | |
| High-temperature stability | A | A | A | A | D |

Based on the results of Table 3, it can be understood that the compositions of Examples 6 and 9 to 11 all had excellent spreadability, caking resistance and high-temperature stability in the evaluation results.

On the other hand, it can be understood that, in Comparative Example 8 in which isostearic acid as a surfactant that was generally used as a dispersing agent was used, the spreadability and high-temperature stability evaluation results were poor.

1. A water-in-oil type emulsion cosmetic composition comprising the following components:
surface-treated zinc oxide particles,
surface-treated titanium oxide particles,
polyhydroxystearic acid,
polyricinoleic acid polyglycerol ester,
an organically modified clay mineral,
an aqueous component, and
a liquid oil,
wherein a content of the polyhydroxystearic acid is less than 1.0 mass %, and
wherein a content of the aqueous component is less than 15 mass %.

2. The composition according to claim 1,
wherein the surface-treated zinc oxide particles are one, two or more selected from among silane coupling-treated zinc oxide, silicone-treated zinc oxide, and sugar fatty acid ester-treated zinc oxide particles.

3. The composition according to claim 2,
wherein the surface-treated zinc oxide particles are one, two or more selected from among triethoxycaprylylsilane-treated zinc oxide particles, dimethicone- and hydrogen dimethicone-treated zinc oxide particles, and dextrin palmitate-treated zinc oxide particles.

4. The composition according to claim 1,
wherein the surface of the surface-treated titanium oxide particles is treated with a fatty acid.

5. The composition according to claim 1,
wherein a content of the surface-treated titanium oxide particles is less than a content of the surface-treated zinc oxide particles, and
wherein a total content of the surface-treated titanium oxide particles and the surface-treated zinc oxide particles is 30 mass % or more.

6. The composition according to claim 1,
wherein the organically modified clay mineral is distearyldimonium hectorite.

7. The composition according to claim 1,
wherein the content of the aqueous component is 10 mass % or less.

8. The composition according to claim 1,
wherein a total content of the polyhydroxystearic acid and the polyricinoleic acid polyglycerol ester is 1.5 to 10 mass %.

9. The composition according to claim 1,
wherein the liquid oil is one, two or more selected from among alkyl fatty acid esters, triglycerides, vegetable oils, and hydrocarbon oils.

10. The composition according to claim 1, further comprising bis-diglyceryl polyacyladipate-2.

11. The composition according to claim 1, which is a sunscreen cosmetic composition.
